(19) United States
(12) Patent Application Publication
(10) Pub. No.: US 2007/0198093 A1
(43) Pub. Date: Aug. 23, 2007

(54) SPINAL IMPLANT WITH OFFSET KEELS

(75) Inventors: Darrel S. Brodke, Salt Lake City, UT (US); John P. Bernero, Round Rock, TX (US)

Correspondence Address: KELLY LOWRY & KELLEY, LLP, 6320 CANOGA AVENUE, SUITE 1650, WOODLAND HILLS, CA, 91367, US

(73) Assignee: AMEDICA CORPORATION, Salt Lake City, UT (US)

(21) Appl. No.: 11/307,701

(22) Filed: Feb. 17, 2006

(51) Int. Cl. A61F 2/44

(52) U.S. Cl. 623/17.15

(57) ABSTRACT

An improved spinal implant includes vertically offset keels for mechanically interlocking respectively with overlying and underlying vertebral bone structures when implanted therebetween. At least one upper keel projects upwardly for close-fit reception into a prepared slot formed in the overlying vertebral structure, and at least one lower keel projects downwardly for similar close-fit reception into a prepared slot formed in the underlying vertebral structure. The upper and lower keels are misaligned or offset from each other to preclude creation of a common stress line extending therethrough, thereby significantly reducing risk of vertebral fracture.

### BACKGROUND OF THE INVENTION

This invention relates generally to improvements in spinal implants such as articulatory and fusion devices of the type designed for human implantation between adjacent spinal vertebrae. More particularly, this invention relates to a spinal implant having upwardly and downwardly projecting keels for improved implant fixation to adjacent spinal vertebrae, wherein these keels are vertically offset or misaligned to safeguard against undesired post-operative fracture of the adjacent vertebral bone structures.

Spinal implant devices are generally known in the art for surgical implantation between adjacent bony vertebral structures to correct or alleviate a number of clinical problems, such as degenerative disc disease, chronic back pain, spondylolisthesis, and others. In general terms, such spinal implants comprise a biocompatible construct formed from a biocompatible polymer, metal or ceramic, with a size and shape for intervertebral placement. In some designs, the implant has a porous region or regions adapted to receive autogenous or allogenous bone material, or otherwise defining a so-called porous bone ingrowth surface, for promoting bone ingrowth attachment of the implant device to the adjacent overlying and underlying vertebral structures. Such spinal implants are commonly used as fusion devices forming a substantially rigid interface between adjacent vertebral structures for alleviating specific patient symptoms. Alternative spinal implant designs comprise articulatory devices including upper and lower components adapted for respective fixation to overlying and underlying vertebral structures while defining an articulatory interface therebetween to maintain or restore normal patient movements.

Many state-of-the-art spinal implants include an upwardly projecting upper keel for relatively close-fit reception into a surgically prepared slot formed in the overlying vertebral structure, in combination with a downwardly projecting lower keel for similar close-fit reception into another surgically prepared slot formed in the underlying vertebral structure. See, for example, U.S. Pat. No. 6,740,118. Such upper and lower keels beneficially provide an improved mechanical interlock with the adjacent vertebrae, to achieve improved fixation of the spinal implant to patient bone. However, such keel structures have been formed on the implant in general vertical alignment with each other, whereby the upwardly and downwardly projecting keels cooperate to define a common stress line coupled with adjoining bone structures along a common line of relative weakness defined by the correspondingly vertically aligned slots cut into the overlying and underlying vertebral structures. As a result, the use of a spinal implant having these keel structures for improved mechanical interlock with patient bone is accompanied by an undesirably increased risk of bone fracture attributable to the common stress line running through the opposed keels and their associated bone slots.

There exists, therefore, a significant need for further improvements in and to spinal implants of the type having upwardly and downwardly projecting keels for improved interlock with adjoining vertebral structures, wherein the risk of post-operative bone fracture along a stress line running through the keel slots is substantially reduced or eliminated. The present invention fulfills these needs and provides further related advantages.

### SUMMARY OF THE INVENTION

In accordance with the invention, an improved spinal implant is provided for implantation between adjacent overlying and underlying vertebral structures for maintaining intervertebral spacing and/or restoring substantially normal intervertebral articulatory function. The improved spinal implant includes at least one upper keel projecting upwardly for relatively close-fit reception into a surgically prepared slot formed in the overlying vertebral structure, and at least one lower keel projects downwardly for similar close-fit reception into a prepared slot formed in the underlying vertebral structure. The upper and lower keels are misaligned or offset from each other, as by vertical misalignment, to preclude creation of a common stress line extending therethrough, thereby significantly reducing or eliminating risk of vertebral fracture.

In one preferred form of the invention, the improved spinal implant includes an upwardly projecting upper keel disposed generally at a centered position on an upper or superior face of the implant, and wherein the upper keel is elongated generally in an anterior-posterior direction. This upper keel is sized and shaped for relatively close-fit reception into a matingly shaped slot formed in the overlying vertebrae as by cutting. The upper keel, when implanted, provides a mechanical interlock with the overlying vertebrae. Such mechanical interlock may be enhanced and/or combined with a porous bone ingrowth surface or holes formed on the upper face of the implant for appropriate fusion-ingrowth with the overlying vertebral structure.

A pair of lower keels project downwardly from a lower or inferior face of the implant, wherein these lower keels are also elongated generally in an anterior-posterior direction. These lower keels are disposed at positions offset from a centered position, or a center axis, of the implant, preferably by positioning these lower keels symmetrically relative to said center axis substantially at the laterally opposed sides of the implant. These lower keels provide a mechanical interlock with the underlying vertebrae, wherein this mechanical interlock may be enhanced and/or combined with a porous bone ingrowth surface or holes formed on the lower face of the implant for appropriate fusion-ingrowth with the underlying vertebral structure.

The thus-constructed spinal implant has the upper and lower keels positioned in offset or misaligned relation, thereby avoiding any common stress line running therebetween or otherwise running between the respective vertebral slots cut into the adjacent vertebral structures for receiving and supporting the keels. As a result, risk of vertebral stress fracture is substantially reduced or eliminated.

Other features and advantages of the invention will become more apparent from the following detailed description, taken in conjunction with the accompanying drawings which illustrate, by way of example, the principles of the invention.

### BRIED DESCRIPTION OF THE DRAWINGS

The accompanying drawings illustrate the invention. In such drawings:

FIG. 1 is a top perspective view of an exemplary articulating spinal implant with offset keels, embodying the novel features of the invention;

FIG. 2 is a bottom perspective view of the spinal implant shown in FIG. 1;

FIG. 3 is a posterior side elevation view of the spinal implant shown in FIG. 1;

FIG. 4 is an elevation view, shown in somewhat schematic form, illustrating intervertebral placement of a plurality of spinal implants with offset keels corresponding with the spinal implant depicted in FIGS. 1-3;

FIG. 5 is a posterior side elevation view similar to FIG. 3, but showing one alternative preferred form of the invention; and

FIG. 6 is an elevation view similar to FIG. 4, shown in somewhat schematic form, illustrating intervertebral placement of a plurality of spinal implants with offset keels corresponding with the spinal implant embodiment depicted in FIG. 5.

### DETAILED DESCRIPTION OF THE PREFERRED EMBODIMENTS

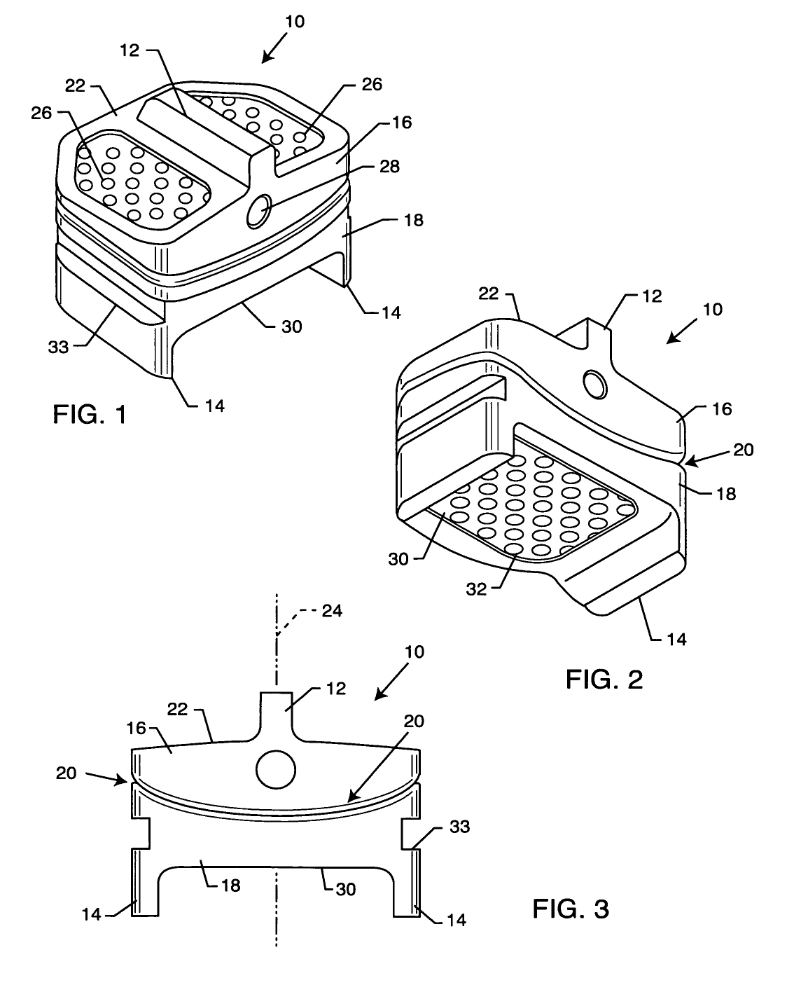

As shown in the exemplary drawings, an improved spinal implant referred to generally in FIGS. 1-4 by the reference numeral 10 is provided for surgical placement between adjacent overlying and underlying bony vertebral structures for maintaining intervertebral spacing and/or restoring substantially normal intervertebral articulatory function. The spinal implant 10 includes at least one upper keel 12 for mechanical interlock with an overlying vertebral structure, and at least one lower keel 14 for similar mechanical interlock with an underlying vertebral structure. In accordance with the invention, the upper and lower keels 12, 14 are disposed in an offset or misaligned relation, as by vertical misalignment, to avoid creating a common vertical stress line extending therethrough, thereby significantly reducing or eliminating risk of vertebral fracture.

The illustrative drawings show the improved spinal implant 10 in one preferred form to comprise an articulatory device having an implant body defined by an upper component 16 adapted for fixation to an overlying vertebral structure, and a lower component 18 adapted for fixation to an underlying vertebral structure. These upper and lower implant components 16, 18 cooperatively define a matingly shaped articulatory interface referred to generally by arrow 20, wherein this articulatory interface is shaped to accommodate a range of inter-component motion consistent with substantially natural intervertebral articulatory function. While the specific geometry of this articulatory interface 20 may vary, preferred configurations are shown and described in U.S. Publi. 2004/0133281, which is incorporated by reference herein. In addition, while the exemplary drawings show the spinal implant 10 in the form of an articulatory device including the upper and lower components 16 and 18, persons skilled in the art will recognize and appreciate that the vertically offset upper and lower keels 12, 14 may be incorporated in a non-articulatory spinal implant of the type designed for intervertebral fusion wherein the upper and lower components 16, 18 are formed as a unitary body.

The upper and lower implant components 16, 18 are constructed from a suitable biocompatible material, such as a titanium-based or a cobalt chrome-based metal, a relatively high strength ceramic, or a biocompatible polymer-based material. Preferred high strength ceramic materials are disclosed in U.S. Pat. No. 6,881,229, which is also incorporated by reference herein. Such ceramic implant may incorporate relatively dense and comparatively porous regions which respectively mimic the physical characteristics of natural cortical and cancellous bone, and wherein the porous region enhances bone ingrowth attachment with the adjoining vertebral structure, as shown and described in U.S. Pat. Nos. 6,790,233 and 6,846,327, which are also incorporated by reference herein.

FIGS. 1-3 show the exemplary articulatory spinal implant 10 wherein the upper component 16 defines an upper or superior face 22 for engagement with the overlying vertebral structure when the implant 10 is surgically placed into the intervertebral space. As shown, this upper face 22 incorporates at least one upper keel 12 in the form of a rigid upstanding rib or ridge protruding upwardly above the plane of the upper face 22. This upper keel 12 is elongated in a fore-aft or anterior-posterior direction, and is substantially centered on a vertical central axis 24 of the implant 10, relative to a left-right or medial-lateral direction. The upper face 22 of the upper component 16 may further incorporate porous bone ingrowth regions 26 (FIG. 1) presented upwardly at opposite sides of the upper keel 12. As previously noted, these bone ingrowth regions may be formed as integral portions of the upper component, as described in U.S. Pat. Nos. 6,790,233 and 6,846,327, or they may be defined as an array of small holes formed as by drilling, or they may comprise porous surface coatings of a type known in the art. A shallow counterbore 28 is shown in the front edge of the upper component 16 for receiving a tip (not shown) of a suitable placement instrument used for intervertebral installation of the implant 10.

The lower component 18 of the spinal implant 10 includes a lower or inferior face 30 for engaging the underlying vertebral structure when the implant is placed into the intervertebral space. As shown, this lower face 30 spans or extends a generally centered region of the lower component 18, between a pair of downwardly protruding lower keels 14 disposed generally at the opposite, or medial and lateral sides of the implant component. These lower keels 14 are also elongated in a fore-aft or anterior-posterior direction, but are disposed in substantially offset or substantially misaligned positions relative to the centered upper keel 12 (and center axis 24) on the upper implant component 16. In the preferred form, the lower keels 14 are positioned in symmetric relation at opposite sides of the center axis 24. The lower face 30 may further incorporate a porous bone ingrowth region 32 (FIG. 2), which may be constructed similar to the ingrowth regions 26 on the upper face 22. In addition, the lower component 18 is shown to include a pair of outboard channels 33 at the opposite sides thereof for engagement with a tool tip (again not shown) of a suitable placement instrument used for intervertebral installation of the implant 10.

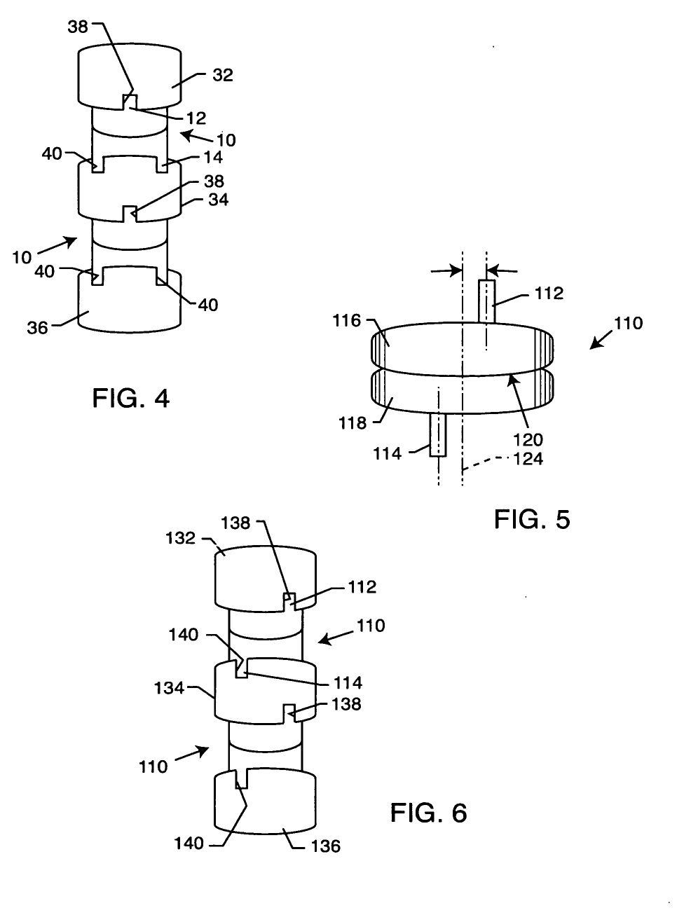

FIG. 4 shows a pair of the spinal implants 10 of FIGS. 1-3 implanted between adjacent vertebrae to maintain vertebral interbody spacing while restoring substantially normal intervertebral articulatory function. More particularly, a first implant 10 is shown between an overlying vertebral structure 32 and an underlying vertebral structure 34, whereas a second implant 10 is shown between the overlying vertebral structure 34 and an adjacent underlying vertebral structure 36. Importantly, prior to intervertebral placement of each spinal implant 10, the adjacent vertebral structures 32, 34 and 34, 36 are surgically prepared as by appropriate surface preparation including cutting of notches for relatively close-fit seated reception of the upper and lower keels 12, 14 on the two implants 10. That is, with reference to the first or upper implant 10, the underside surface of the overlying vertebral structure 32 is prepared by cutting a notch 38 extending generally in a fore-aft or anterior-posterior direction, with a size and a position for relatively close-fit reception of the vertically centered upper keel 12 on the implant upper component 16. Similarly, the upwardly presented surface of the underlying vertebral structure 34 is prepared by cutting a laterally spaced pair of notches 40 with a size and position for relatively close-fit reception of the off-centered lower keels 14 on the implant lower component 18. For the second or lower implant 10, the surfaces of the vertebral structures 34 and 36 are similarly prepared to define notches 38 and 40 for respectively receiving the upper and lower keels 12 and 14.

While the illustrative drawings show a pair of the spinal implants 10 installed between adjacent pairs of vertebral structures, persons skilled in the art will understand that one or more of the spinal implants 10 may be employed according to the condition of each specific patient.

Post-surgically, each spinal implant 10 maintains appropriate intervertebral spacing while, in the case of the illustrative articulatory implant device, restoring substantially normal patient movement function. Importantly, the upper and lower keels 12, 14 of each spinal implant 10 are vertically out-of-alignment, so that there is no common vertical stress line extending between the overlying and underlying vertebral structures. Similarly, when multiple implants 10 are placed between adjacent pairs of vertebrae, the lower keels 14 of an upper implant are also vertically misaligned with the upper keel 12 of a lower implant, thereby also avoiding any common vertical stress line extending through any single vertebral structure such as the intermediate vertebrae 34 shown in FIG. 4. The absence of such vertical stress line through the keels 12,14 and the associated keel notches 38, 40 significantly reduces and substantially eliminates risk of stress fracture of the adjoining vertebral structures.

FIGS. 5 and 6 depict one preferred alternative form of the invention, wherein components corresponding in structure and/or function with those previously shown and described in FIGS.1 -4 are identified by common reference numerals increased by 100. As shown, a modified spinal implant 110 includes a single upstanding upper keel 112 and a single downwardly projecting lower keel 114, wherein both keels 112, 114 are positioned in offset relation to each other and with respect to a vertical center axis 124 of the implant.

More particularly, FIG. 5 shows the modified spinal implant 110 in the form of an articulatory device having an upper component 116 and a lower component 118 defining an articulator interface 120. The upper keel 112 projects upwardly from the upper component 116 at a location off-axis relative to the implant center axis 124, whereas the lower keel 114 projects downwardly from the lower component 118 at a location which is also disposed off-axis relative to the center axis 124 as further disposed off-axis relative to the upper keel 112. In the preferred geometry as shown, the upper keel 112 and the lower keel 114 are positioned off-axis in opposite directions in the medial-lateral direction relative to the center axis 124, and preferably in a manner positioning the upper and lower keels 112, 114 substantially symmetric to the center axis 124.

FIG. 6 shows a pair of the modified spinal implants 110 installed between adjacent vertebrae to maintain vertebral interbody spacing while restoring substantially normal intervertebral articulatory function. More particularly, a first implant 110 is shown between an overlying vertebral structure 132 and an underlying vertebral structure 134, whereas a second implant 110 is shown between the overlying vertebral structure 134 and an adjacent underlying vertebral structure 136. Prior to intervertebral placement of each spinal implant 110, the adjacent vertebral structures 132, 134 and 134,136 are surgically prepared as by appropriate surface preparation including cutting of notches for relatively close-fit seated reception of the upper and lower keels 112, 114 on the two implants 110. That is, with reference to the first or upper implant 110, the underside surface of the overlying vertebral structure 132 is prepared by cutting a notch 138 extending generally in a fore-aft or anterior-posterior direction, with a size and a position for relatively close-fit reception of the offset upper keel 112 on the implant upper component 116. Similarly, the upwardly presented surface of the underlying vertebral structure 134 is prepared by cutting a notch 140 with a size and position for relatively close-fit reception of the off-centered lower keel 114 on the implant lower component 118. For the second or lower implant 110, the surfaces of the vertebral structures 134 and 136 are similarly prepared to define notches 138 and 140 for respectively receiving the upper and lower keels 112 and 114.

Post-surgically, each modified spinal implant 110 maintains appropriate intervertebral spacing while, in the case of the illustrative articulatory implant device, restoring substantially normal patient movement function. Importantly, the upper and lower keels 112, 114 of each spinal implant 110 are, once again, positioned in vertical misalignment, so that there is no common vertical stress line extending between the overlying and underlying vertebral structures. Similarly, when multiple implants 110 are placed between adjacent pairs of vertebrae, the lower keels 114 of an upper implant are also vertically misaligned with the upper keel 112 of a lower implant, thereby also avoiding any common vertical stress line extending through any single vertebral structure such as the intermediate vertebrae 134 shown in FIG. 6. The absence of such vertical stress line through the keels 12, 114 and the associated keel notches 138, 140 significantly reduces and substantially eliminates risk of stress fracture of the adjoining vertebral structures.

A variety of further modifications and improvements in and to the improved spinal implant of the present invention will be apparent to those persons skilled in the art. In this regard, while the invention is shown and described herein with at least one upper keel in vertical misalignment with at least one lower keel, persons skilled in the art will appreciate that the orientation terms “upper”, “lower”, and “vertical” are exemplary only and that alternative keel misalignment orientations including but not limited to fore-aft or medial-lateral misalignment are within the scope of the disclosed invention. Accordingly, no limitation on the invention is intended by way of the foregoing description and accompanying drawings, except as set forth in the appended claims.

What is claimed is:

1. A spinal implant for implantation between adjacent vertebrae, comprising: an implant body having at least one upper keel protruding upwardly therefrom for engaging an overlying vertebral structure and at least one lower keel protruding downwardly therefrom for engaging an underlying vertebral structure, said upper and lower keels being positioned in offset relation to each other.

2. The spinal implant of claim 1 wherein said implant body comprises an upper component and a lower component cooperatively defining an articulatory interface therebetween, said at least one upper keel protruding upwardly from said upper component and said at least one lower keel protruding downwardly from said lower component.

3. The spinal implant of claim 1 wherein said implant body defines an upper face including at least one bone ingrowth region thereon, said at least one upper keel protruding upwardly from said upper face, and further wherein said implant body defines a lower face including at least one bone ingrowth region thereon, said at least one lower keel protruding downwardly from said lower face.

4. The spinal implant of claim 1 wherein said at least one upper keel comprises a rib projecting upwardly from said implant body and disposed generally on a vertical center axis of said implant body, and further wherein said at least one lower keel comprises a pair of ribs projecting downwardly from said implant body and respectively disposed in offset relation at opposite sides of said vertical center axis.

5. The spinal implant of claim 4 wherein said pair of ribs projecting downwardly from said implant body are disposed generally symmetrically with respect to said vertical center axis.

6. The spinal implant of claim 1 wherein said at least one upper keel comprises a rib projecting upwardly from said implant body and disposed in offset relation to one side of a vertical center axis of said implant body, and further wherein said at least one lower keel comprises a rib projecting downwardly from said implant body and disposed in offset relation to said upper keel and at an opposite side of said vertical center axis.

7. The spinal implant of claim 1 wherein said ribs defining said upper and lower keels are disposed generally symmetrically with respect to said vertical center axis.

8. A spinal implant for implantation between adjacent vertebrae, comprising: an implant body having an upper keel protruding upwardly therefrom generally on a vertical center axis thereof for engaging a corresponding notch formed in an overlying vertebral structure, and a pair of lower keels protruding downwardly therefrom generally in offset relation to and at opposite sides of said vertical center axis for engaging a pair of corresponding notches formed in an underlying vertebral structure, said upper and lower keels being positioned in misaligned offset relation to each other.

9. The spinal implant of claim 8 wherein said implant body comprises an upper component and a lower component cooperatively defining an articulatory interface therebetween, said upper keel protruding upwardly from said upper component and said lower keels protruding downwardly from said lower component.

10. The spinal implant of claim 8 wherein said implant body defining an upper face including at least one bone ingrowth region thereon, said upper keel protruding upwardly from said upper face, and further wherein said implant body defines a lower face including at least one bone ingrowth region thereon, said lower keels protruding downwardly from said lower face.

11. A spinal implant for implantation between adjacent vertebrae, comprising: an implant body having an upper keel protruding upwardly therefrom generally in offset relation to a center axis thereof for engaging a corresponding notch formed in an overlying vertebral structure, and a lower keel protruding downwardly therefrom generally in offset relation to said vertical center axis thereof for engaging a corresponding notch formed in an underlying vertebral structure, said lower upper and lower keels being positioned in misaligned offset relation to each other and at opposite sides of said center axis.

12. The spinal implant of claim 11 wherein said implant body comprises an upper component and a lower component cooperatively defining an articulatory interface therebetween, said upper keel protruding upwardly from said upper component and said lower keel protruding downwardly from said lower component.

13. The spinal implant of claim 11 wherein said implant body defines an upper face including at least one bone ingrowth region thereon, said upper keel protruding upwardly from said upper face, and further wherein said implant body defines a lower face including at least one bone ingrowth region thereon, said lower keel protruding downwardly from said lower face.

14. The spinal implant of claim 11 wherein said upper and lower keels are disposed generally symmetrically with respect to said center axis.
